(19) United States
(12) Patent Application Publication
(10) Pub. No.: US 2022/0047756 A1
(43) Pub. Date: Feb. 17, 2022

(54) DISTRIBUTION CAROUSEL

(71) Applicant: FAMILY SELF CARE, Paris (FR)

(72) Inventors: Brigitte THITO, Paris (FR); Christophe EVERARTS, Paris (FR)

(21) Appl. No.: 17/297,786

(22) PCT Filed: Nov. 30, 2018
(86) PCT No.: PCT/FR2018/053065

§ 371 Date: May 27, 2021

(51) Int. Cl. A61L 9/12

(52) U.S. Cl. CPC A61L 9/125; A61L 2209/131; A61L 2209/133

(57) ABSTRACT

The distribution carousel (1) comprising a body (10) comprises a set of receptacles (13) of a set of reservoirs (20, 21) for fluid containing a fluid that is to be distributed, each one of the reservoirs comprising a distribution orifice (22) having a distribution axis (X), the set of receptacles being arranged in the body such that, once the reservoirs of the set of reservoirs are in place in the associated receptacles, the distribution axes of the reservoirs of the set of reservoirs intersect in a service region (Z) around a single mixing point (P).

### TECHNICAL FIELD OF THE INVENTION

The invention relates to a carousel for distributing products, in particular fluids and especially essential or vegetable oils.

### PRIOR STATE OF THE ART

A certain number of fluids such as aromatic fluids, for example essential oils, are distributed in small reservoirs, also called vials: a vial or reservoir containing a specific essential oil. Document WO90/03192, for example, describes such small reservoirs, intended to contain and keep such aromatic fluids, said small reservoirs having a cylindrical shape containing, in the lower portion, a distribution orifice distributing a calibrated drop of aromatic fluid and, at their upper extremity, a cap made of a flexible elastomer, elastically deformable so as to form a drip-feed device. In this document, the different reservoirs are positioned in receptacles of a carousel in an aligned manner. A container is positioned below the set of reservoirs to receive the drops of essential oils from these reservoirs. However, during distribution, the drops fall into the container separated from each other. Consequently, a mixing device is needed to produce a mixture of these drops. This has a drawback of making the production of such essential oil mixtures more complex and imposes a possible requirement of cleaning and sterilizing the mixing device.

### DESCRIPTION OF THE INVENTION

One aim of the invention is to provide a distribution carousel that makes it possible to produce compositions based on mixtures in a simple way without having the drawbacks presented above.

To this end, the invention envisages a distribution carousel, comprising a body that comprises a set of receptacles of a set of reservoirs for fluid containing a fluid that is to be distributed, each one of the reservoirs comprising a distribution orifice having a distribution axis, the set of receptacles being arranged in the body such that, once the reservoirs of the set of reservoirs are in place in the associated receptacles, the distribution axes of the reservoirs of the set of reservoirs intersect in a service region around a single mixing point.

Advantageously, but optionally, the carousel according to the invention has at least one of the following technical characteristics:
the mixing point and the service region are located under the body, while the carousel is in use;
each one of the receptacles of the set of receptacles forms a reservoir of the set of reservoirs for fluid;
the body extends linearly;
the body broadly has a shape of revolution;
the set of receptacles comprises a central receptacle and peripheral receptacles distributed along a circumference around the central receptacle;
the carousel also comprises a protective cover covering the set of receptacles;
the carousel comprises a base;
the base comprises a container whose bottom is positioned at the location of the mixing point or service region;
the container is removable;
the cover forms the removable container; and
the container is attached removably on the bottom of the body.

### BRIEF DESCRIPTION OF THE FIGURES

Other features and advantages of the invention will become apparent from reading the description that follows of an embodiment of the invention. In the drawings included in an appendix:

FIG. 1 is a three-dimensional view of an embodiment of the distribution carousel according to the invention;

FIG. 2 is a side view of the carousel of FIG. 1;

FIG. 3 is a top view of the carousel of FIG. 1;

FIG. 4 is a cross-section view of the carousel of FIG. 1; and

FIG. 5 is a cross-section view of an embodiment variant of the carousel of FIG. 1.

For greater clarity, identical or similar elements are identified by identical reference signs in all the figures.

### Detailed Description of an Embodiment

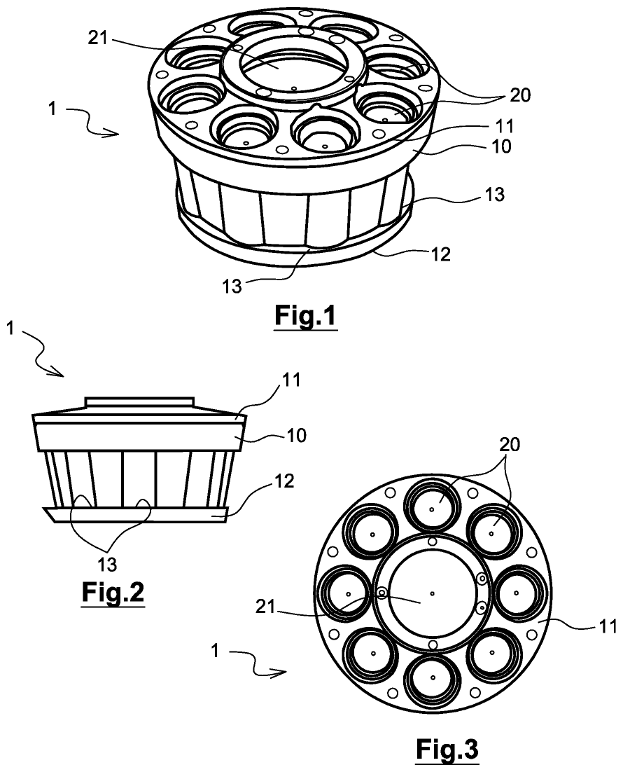

With reference to FIGS. 1 to 4, we will describe an embodiment of the distribution carousel 1 according to the invention.

The distribution carousel 1 according to the invention comprises a body 10, here broadly has a shape of revolution, in particular frustoconical. The body 10 comprises an upper cover 11 and a bottom 12. A set of receptacles 13 is fitted in the body 10. Shown here, the set of receptacles comprises a central receptacle and a set of peripheral receptacles arranged along a circumference. In the figures, there are eight peripheral receptacles 13 uniformly distributed over the circumference around the central receptacle, which is larger than the peripheral receptacles 13. The receptacles 13 are delimited in the lower portion by the bottom 12 and in the upper portion by the cover 11. According to some variant embodiments, each of the receptacles is a hollow volume with a specific shape, for example a revolution shape, parallelepipedal or polygonal, delimited over the entire length of the section of the body 10 by the material forming the body 10.

The receptacles 13 of the set of receptacles 13 are arranged such that each receives a reservoir 20, 21 for fluid of the set of reservoirs 20, 21 for fluid. Here, the reservoirs 20, 21 for fluid are reservoirs containing essential or vegetable oils or other fluids of different viscosities: each reservoir 20, 21 contains an essential or vegetable oil or a different fluid. The reservoirs 20, 21 are, for example, standard vials, known per se, with a revolution shape, for example cylindrical or frustoconical; an inverted frustoconical shape has the advantage of being able to optimize the useful volume. They comprise in the lower portion a distribution orifice 22 which has a distribution axis X. In the upper portion, the reservoirs 20, 21 are closed by a membrane (not shown) in a way known per se. Each reservoir 20,21 is received in an associated receptacle 13 and is secured to it by various known means, for example, abutted against the bottom 12.

In a variant embodiment, the reservoirs 20, 21 are dual chamber reservoirs like those described, for example, in document EP18305576, which may be referred to for more detailed information.

In another variant embodiment, the receptacles 13 form the reservoirs 20, 21, which are then incorporated directly into the distribution carousel 1 according to the invention.

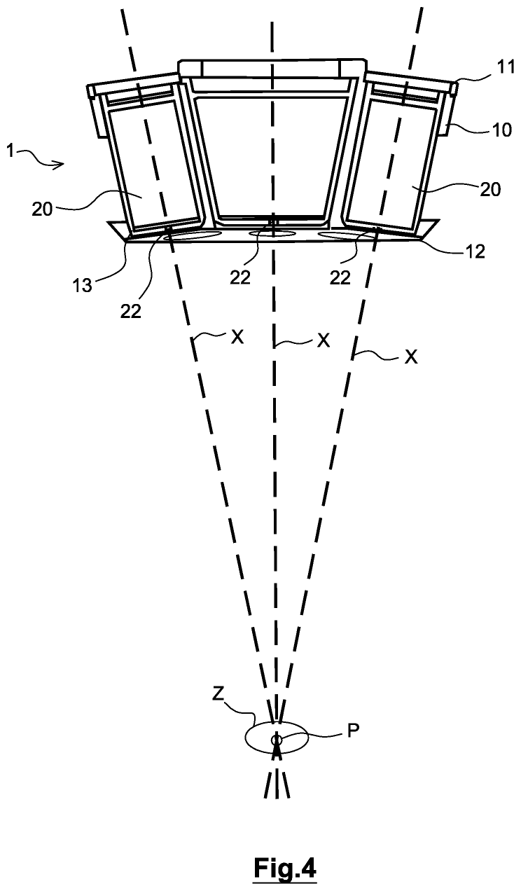

As shown in FIG. 4, the receptacles 13 of the set of receptacles 13 are oriented such that, once the reservoirs 20, 21 are installed in their associated receptacle 13, the distribution axes X of said reservoirs 20, 21 intersect two by two in a point located in a service region Z corresponding to a volume delimited around a point P, referred to as a single mixing point; for example, the service region Z is delimited by a sphere or an ovoid volume having a largest dimension of between approximately 1 and 10 millimeters, preferably less than 1 mm. Optimally, the distributions axes X of said reservoirs 20, 21 intersect at the single mixing point P. In this way, a mixture of different essential or vegetable oils distributed through the distribution orifices 22 of the reservoirs 20, 21 is produced at the location of the single mixing point P or the service region Z in a natural way without requiring a device constituting a mixer. In this way, the distribution carousel 1 according to the invention makes it possible to dispense with such a mixing device, which normally requires additional operations while introducing a requirement of cleaning and sterilization between two mixtures. Note that the single mixing point P, or the service region Z, is positioned opposite to and at a distance from the bottom 12 of the body 10 of the distribution carousel 1 according to the invention: the single mixing point P, or the service region Z, is located under the body 10, while the distribution carousel 1 according to the invention is in use.

According to a variant embodiment, the distribution carousel 1 according to the invention comprises a body 10 which has an elongated shape: the body 10 thus extends linearly, the receptacles 13 being positioned in line next to one another along the body 10.

Optionally, the distribution carousel 1 according to the invention also comprises a protective cover 15 covering the set of receptacles 13. For this, the protective cover 15 is installed on the upper portion of the body 10 so as to extend opposite the cover 11. The protective cover 15 can be mounted in a removable or articulated way on the body 10, thus allowing access to the reservoirs 20, 21 to replace them individually, in the case where these reservoirs 20, 21 are received in the receptacles 13. In a variant embodiment, the protective cover 15 is mounted in an irremovable way on the body 10 of the distribution carousel 1 according to the invention, for example by crimping. In all cases, one role of the protective cover 15 is to protect the upper portion, i.e. the membrane, of the reservoirs 20, 21, when the distribution carousel 1 according to the invention is being handled by a user, thereby preventing any inadvertent contact on the membrane that would trigger an unintended distribution of a portion of the contents of the associated reservoir.

The distribution carousel 1 according to the invention that has just been described is intended to be inserted into a distribution machine.

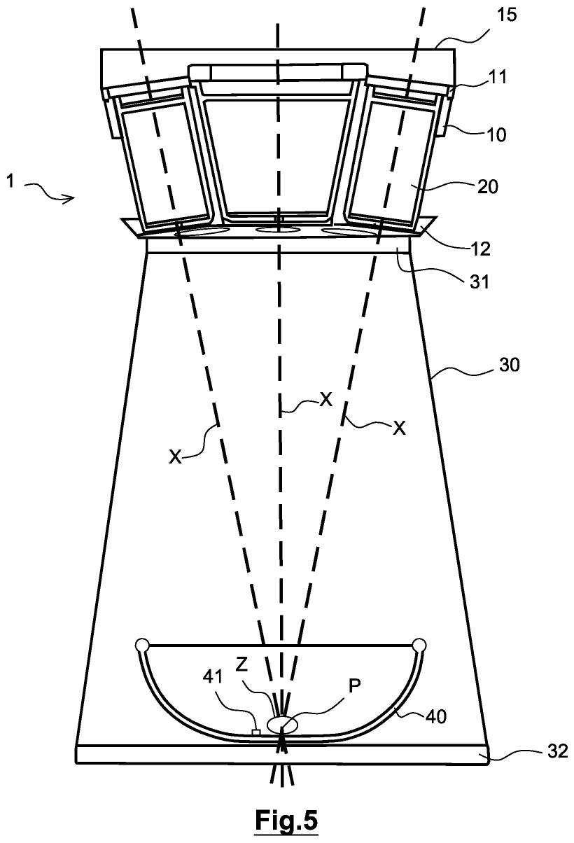

According to another variant embodiment, illustrated in FIG. 5, the distribution carousel 1 according to the invention comprises a secured base 30 that makes it possible to stabilize the distribution carousel 1 according to the invention, when in use: this enables direct manual use of the distribution carousel 1 according to the invention. The base 30 comprises a support 31 for receiving the body 10. In particular, the support 31 cooperates with the bottom 12 for this purpose. In addition, the base 30 comprises a baseplate 32 located at a distance from the support 31 and connected rigidly to it.

According to a variant embodiment, the carousel 1 and the secured base 30 are attached to each other in a removable way (for example by clipping). This advantageously makes it possible to replace a carousel 1 according to the invention, for example if the reservoirs 20, 21 are empty and if the carousel 1 is secured to reservoirs 20, 21 (the receptacles 13 thus forming the reservoirs 20, 21), by another carousel of the same type while using the same secured base 30.

The distribution carousel 1 according to the invention comprises a container 40 intended to receive, while in use, a mixture of fluids from reservoirs 20, 21. For this, the container 40 is positioned such that a bottom 41 of the container is placed at the location of the single mixing point P or the service region Z. Here, the container 40 is incorporated in the base 30. In a variant, the container 40 is removable. In another variant, the removable version of the protective cover 15 forms the container 40. In an additional variant, the container 40 is attached removably on the bottom 12 of the body 10 of the distribution carousel 1 according to the invention, for example by clipping or screwing.

Of course, it is possible to make many modifications to the invention without in any way leaving the scope of the invention.

1. A distribution carousel comprising a body comprising a set of receptacles of a set of reservoirs for fluid, said reservoirs containing a fluid that is to be distributed, each one of the reservoirs comprising a distribution orifice having a distribution axis, wherein the set of receptacles is arranged in the body such that, once the reservoirs of the set of reservoirs are in place in the associated receptacles, the distribution axes of the reservoirs of the set of reservoirs intersect in a service region around a single mixing point.

2. The carousel according to claim 1, wherein the mixing point and the service region are located under the body, while the carousel is in use.

3. The Carousel according to claim 1 wherein each one of the receptacles of the set of receptacles forms a reservoir of the set of reservoirs for fluid.

4. The carousel according to claim 1, wherein the body extends linearly.

5. The carousel according to claim 1, wherein the body broadly has a shape of revolution.

6. The carousel according to claim 5, wherein the set of receptacles comprises a central receptacle and peripheral receptacles distributed along a circumference around the central receptacle.

7. The carousel according to claim 1, characterized in that the carousel also comprises a protective cover covering the set of receptacles.

8. The carousel according to claim 1, wherein the carousel comprises a base.

9. The carousel according to claim 1, wherein the carousel comprises a container whose bottom is positioned at the location of the mixing point or service region, while in use.

10. The carousel according to claim 9, characterized in that the container is removable.

11. The carousel according to claim 7, wherein the cover forms the removable container.

12. The carousel according to claim 10, characterized in that the container is attached removably on the bottom of the body of the carousel.
